(19) United States
(12) Patent Application Publication
(10) Pub. No.: US 2026/0310774 A1
(43) Pub. Date: Oct. 8, 2026

(54) Driver Identification

(71) Applicant: Cambridge Heartwear Ltd, Cambridge (GB)

(72) Inventors: Samarakoon Mudiyanselage Chatura Theekshana Bandar Samarakoon, Cambridge (GB); Rameen Shakur, Cambridge (GB)

(21) Appl. No.: 19/713,088

(22) Filed: Jun. 18, 2026

Related U.S. Application Data
(62) Division of application No. 18/986,124, filed on Dec. 18, 2024.

(51) Int. Cl. G06F 21/32; B60R 25/25; B62D 1/04

(52) U.S. Cl. CPC G06F 21/32; B60R 25/25; B62D 1/046

(57) ABSTRACT

A computer implemented method of determining an identity of a driver is provided. The method comprises: obtaining a first electrocardiogram, ECG, signal for an authorised driver; providing at least part of the first ECG signal to a machine learning algorithm to obtain a first vector representation of features of the first ECG signal; labelling the first vector representation as belonging to the authorised driver, the labelling being based upon an identity token provided by the authorised driver prior to or at the time of obtaining the first ECG signal; obtaining a second ECG signal for a candidate driver; providing at least part of the second ECG signal to the machine learning algorithm to obtain a second vector representation of features of the second ECG signal; comparing the second vector representation to the labelled first vector representation to determine a similarity metric; and if the similarity metric is within a predetermined threshold, providing confirmation that the candidate driver is the authorised driver.

### CROSS-REFERENCE TO RELATED APPLICATIONS

This is a divisional application of U.S. patent application Ser. No. 18/986,124 filed on Dec. 18, 2024, which is incorporated by reference.

### FIELD OF THE INVENTION

The present invention relates to methods and systems for identifying drivers based at least in part on electrocardiogram (ECG) measurements.

### BACKGROUND

Electrocardiogramique that is widely used for variety of medical and general fitness and health-based purposes. In clinical settings, highly specialised ECG systems are able to precisely measure the activity of the heart and therefore detect and monitor a number of cardiovascular conditions. In non-clinical settings, ECG can be utilised in wearable technologies and other devices to provide a convenient means for monitoring heart activity.

A person's individual ECG may be unique to them and may therefore be regarded as a biometric signature. By recording an ECG signal for a person and analysing it, it may be possible to identify a person based upon their individual ECG. However, such identification is generally difficult to perform in real-world, non-clinical settings. ECG signals obtained using commercial devices, particularly those where electrodes are not placed at optimal locations such as the chest, are typically small in amplitude. Therefore, such ECG signals are easily obscured by general noise and fluctuations outside of clinical settings. This means that it may be difficult to extract and analyse any useful information from the ECG signals that may be used for identification or other classification.

However, because ECG is a powerful technique for analysing the human body, there is great interest in incorporating ECG systems into a wide range of products and scenarios. Thus, there is a desire to address at least some of the difficulties associated with practically integrating ECG into real-world devices.

### SUMMARY

According to a first aspect, there is provided a computer implemented method of determining an identity of a driver. The method comprises: obtaining a first electrocardiogram, ECG, signal for an authorised driver; providing at least part of the first ECG signal to a machine learning algorithm to obtain a first vector representation of features of the first ECG signal; labelling the first vector representation as belonging to the authorised driver, the labelling being based upon an identity token provided by the authorised driver prior to or at the time of obtaining the first ECG signal; obtaining a second ECG signal for a candidate driver; providing at least part of the second ECG signal to the machine learning algorithm to obtain a second vector representation of features of the second ECG signal; comparing the second vector representation to the labelled first vector representation to determine a similarity metric; and if the similarity metric is within a predetermined threshold, providing confirmation that the candidate driver is the authorised driver.

The method may generally be regarded as an ECG onboarding process that makes use of existing authorisation tokens to establish an ECG ‘profile’ for drivers, against which ECG-based identification can then be performed. The authorised driver is regarded as an authorised driver because they are in possession of the identity token. The identity token may be, for example, a mobile phone comprising an application, the application containing details that identify the authorised driver as being a person X. The first ECG signal may be obtained when the authorised driver first uses a vehicle, during an initial onboarding process. Using the machine learning algorithm, the first ECG signal is analysed, and the first vector representation obtained. Because the identity token has been provided, it is known that the first vector representation (e.g., an embedding vector of ECG signal features output by the machine learning model) corresponds to an ECG signal obtained from the authorised driver/person X. Thus, the first vector representation may be regarded as an ECG profile for person X.

Subsequently, a candidate driver provides a second ECG signal. In the same way as for the first ECG signal, the second ECG signal is analysed by the machine learning algorithm in order to produce a second vector representation. This is compared to the first vector representation in order to determine a similarity between the two. In other words, the ECG profile of the candidate driver is compared to the ECG profile of the authorised driver. Where the candidate driver is also person X, the vector representations will be sufficiently similar to enable a positive confirmation that the candidate driver is the authorised driver. However, where the candidate driver is a new person Y, the vector representations will not be sufficiently similar and thus the method determines that the candidate driver is not the authorised driver.

The ability to determine the identity of drivers based upon ECG signals can enable a number of potential advantages. Accurate, ECG-based identification may allow for further methods and operations to be performed based upon the determined driver identity. The identification could be used as part of a security protocol. For example, only authorised drivers may be allowed to operate a vehicle or perform certain actions in relation to the vehicle. Once the ECG-based identification is sufficiently optimised (as discussed further below), the authorised driver may be authorised to operate the vehicle based upon the ECG identification alone, without the need to provide the identity token that is normally required. Additionally, or alternatively, the identification could be used to automatically provide individual driver preferences. For example, vehicle settings such as audio/infotainment settings, navigation options, seating position or driver aid options may be automatically adjusted to predetermined settings based upon the determined driver identity.

Additionally, the ability to determine driver identity may allow for further or more accurate analysis of the ECG signal itself. For example, algorithms may be applied to the obtained ECG signals to determine particular properties of the ECG signal. These properties could relate to cardiovascular related health conditions (e.g., atrial fibrillation or arrythmia) and/or to physiological conditions (e.g., tiredness, alcohol intoxication or drug use) which may present themselves as an atypical ECG signal. Any suitable algorithm for analysing ECG signals to identify cardiovascular or physiological conditions may be used in conjunction with the identification method discussed herein. But in any case, the determined identity of the driver may be used to inform or modify an outcome of said analysis. For example, the obtained ECG signal may be analysed for a particular heart condition. If such said heart condition is detected, a notification may be provided to the driver alerting them to the fact that an abnormal ECG signal has been detected. However, if it is known that the identified driver has said heart condition (or a heart condition that results in similar ECG signal features), based on previous ECG signals or on additional information previously provided by the driver, then it may not be necessary to provide a notification.

The first ECG signal may be obtained for a longer time duration than the second ECG signal and may comprise a plurality of ECG signal segments taken at different times. Each of the plurality of ECG signal segments of the first ECG signal may be provided to the machine learning algorithm individually, so as to produce plurality of first vector representations. The plurality of first vector representations may be combined to form a first vector representation space that is indicative of the features of the plurality of ECG signal segments.

The first ECG signal may be obtained across a duration of more than 30 seconds, more than 1 minute, more than 5 minutes or more than 10 minutes, for example. For example, the driver may be instructed to place a body part, (e.g., their palm, fingers and/or thumbs) on one or more ECG electrodes for 10 to 15 minutes. During this time, the driver may be instructed to move or reposition their fingers on the ECG electrodes (for example to improve an electrical contact with the electrodes). The ECG signal that is obtained during this time may then be divided into a plurality of segments, each of which may then be analysed individually using the machine learning algorithm. The first vector representations may then be combined using any suitable mathematical function, so as to form the vector representation space. Each first vector representation will have a magnitude in each dimension of the vector. Combining the first vector representations may comprise calculating an average of said magnitudes in each dimension. The first vector representation space may then be defined by the variance in the first vector representations around this average. The first vector representation space may cover a volume extending one standard deviation (or multiple thereof) from the average in each dimension, for example.

The first vector representation space may thus define a more generalised ECG profile for the authorised driver. Rather than being just a single point within the vector space (i.e., a single first vector representation), the first vector representation space covers a volume that reflects the variability in the authorised driver's ECG signal. The second vector representation for the candidate driver can then be compared to this more generalised first vector representation space to determine a similarity; e.g., does the second vector representation fall within the first vector representation space or within a predetermined distance of it.

If the similarity metric is outside of the predetermined threshold, the method may further comprise: retrieving an identity token provided by the candidate driver prior to or at the time of obtaining the second ECG signal; comparing the retrieved identity token provided by the candidate driver to the identity token provided by the authorised driver; and if the identity token provided by the candidate driver corresponds to the identity token provided by the authorised driver, combining the second vector representation with the plurality of first vector representations, thereby updating the first vector representation space.

Where it is determined that the second vector representation is not sufficiently similar to the first vector representation (i.e., the candidate driver is not recognised as the authorised driver based upon the ECG signals), the method may fall back upon the alternative identity tokens to perform the identification, assuming that the candidate driver has an identity token. Where the identity token provided by the candidate driver matches the identity token provided by the authorised driver, it may be assumed that the candidate and authorised driver are in fact the same person despite the ECG comparison. The identity token provided by the authorised driver may be stored in a memory for said comparison. The second vector representation may be combined with the first vector representation similar to above (e.g., the first and second vector representations may be combined to form the first vector representation space). Thus, the ECG profile of the authorised driver can be updated based upon subsequent ECG signals; the vector representation space can be iteratively adapted to provide a more accurate profile and better reflect any variability in ECG features of the driver. This may be particularly advantageous while the authorised driver is still relatively new to a vehicle and their ECG profile is not yet fully established. This process may be repeated each that the authorised driver provides further ECG signals that are not sufficiently similar to the existing first vector representation space.

The method may further comprise: obtaining a third ECG signal for a further candidate driver; providing at least part of the third ECG signal to the machine learning algorithm to obtain a third vector representation of features of the third ECG signal; comparing the third vector representation to the first vector representation space to determine the similarity metric; and if the similarity metric is within a predetermined threshold, providing confirmation that the further candidate driver is the authorised driver.

Following the iterative updating of the authorised driver's ECG profile (e.g., repeated updating of the first vector representation space), ECG signals of further candidate drivers may be obtained, analysed using the machine learning algorithm and compared to the existing first representation space. If the similarity metric is within a predetermined threshold, then positive confirmation that the further candidate driver matches the authorised driver can be provided. If the similarity metric is within a predetermined threshold, then the iterative updating of the first vector representation space discussed above can be repeated for the further candidate driver.

If the identity token provided by the further candidate driver does not correspond to the identity token provided by the authorised driver, the method may further comprise: labelling the third vector representation as belonging to the further candidate driver, the labelling being based upon the identity token provided by the candidate driver; and storing the labelled third vector representation as belonging to a second authorised driver, the further candidate driver being the second authorised driver.

Where vector representations of candidate drivers do not match an existing authorised driver, and/or where provided identity tokens confirm that the candidate driver is not the same person as the authorised driver, then the candidate driver may be added as an additional authorised driver. That is, the non-matching candidate driver may be regarded as a new authorised driver and an ECG profile established accordingly. The non-matching candidate driver may be added as an authorised driver in the same way as the aforementioned authorised driver was added; i.e., using the initial onboarding process discussed above. This process may be repeated each time a candidate driver that does not have a matching vector representation or matching identity token presents themselves. Each individual person may be added as a new vector representation space/ECG profile using the initial onboarding process.

The second ECG signal may comprise a plurality of ECG signal segments taken at different times. Each of the plurality of ECG signal segments of the second ECG signal may be provided to the machine learning algorithm individually, so as to produce plurality of second vector representations. The plurality of second vector representations may be combined to form a second vector representation space that is indicative of the features of the plurality of ECG signal segments.

In the same way that the first ECG may comprise a plurality of segments, from which a plurality of vector representations are obtained and combined, the same can be done for the second ECG signal. Forming a second vector representation space—i.e., combining multiple second vector representations—may mean that any noise or fluctuations in the second ECG signal may be averaged out. Thus, the second vector representation space may constitute a more reliable ECG profile of the candidate driver, thus allowing for more accurate driver identification.

The method may further comprise applying a feature extraction algorithm to the first ECG signal, the feature extraction algorithm being configured to identify the position of at least one of a P, Q, R, S and T peak of the first ECG signal. Providing at least part of the first ECG signal to a machine learning algorithm may comprise providing the positions of the identified at least one P, Q, R, S and T peak of the first ECG signal to the machine leaning algorithm.

At least one of a P, Q, R, S and T peak may comprise, for example, a plurality of at least one of P, Q, R, S, and T peaks. Not all beats well necessarily have each feature present.

The feature extraction algorithm may be configured to identify each of the P, Q, R, S and T peak in each ECG beat (including their location and size within the ECG signal) and identify the location and duration of a corresponding PR, ST and QT interval, for example. Although described here in relation to the first ECG signal, the same feature extraction process may be applied to all ECG signals that are analysed using the methods herein.

The locations of each peak and any corresponding intervals/segments that are determined may be provided as inputs to the machine learning (ML) algorithm for determining the vector representation. Using the extracted features as inputs may reduce the number of computational resources that are required to produce the vector representation, while still retaining sufficient information for characterising ECG signals. The location and size of the peaks may comprise most of the useful information within an ECG and may therefore provide suitable features for subsequent analysis. Using these extracted features as inputs for the ML algorithm may therefore reduce noise/variability in the determined vector representations that form the ECG profiles. Thus, the method may provide for more accurate or reliable driver authentication based upon comparison of said vector representations. Alternatively, the entire ECG signal may be provided as an input to the machine learning algorithm.

The feature extraction algorithm may be a machine learning-based algorithm, the feature extraction machine learning algorithm having been trained using labelled training ECG data to identify peaks within ECG signals.

The feature extraction algorithm may comprise a convolutional neural network (CNN) architecture, for example. The feature extraction algorithm may be according to the methods and systems described in WO 2019/234458 A1, but it will be understood that any suitable ECG feature extraction algorithm or process may be applied.

The method may further comprise: for each ECG signal, measuring the heart rate of the driver based upon features of the ECG signal; sampling each ECG signal, the rate of sampling being proportional to the heart rate measured for said ECG signal.

Sampling the ECG signal at a rate proportional to the heart rate of the driver may improve the accuracy with which the vector representations are determined and also the accuracy with which the vector representations are compared to one another. The comparison of authorised and candidate driver ECG signals/vector representations should be heart rate invariant; a difference in heart rate should not impact the driver identification process. Varying the sample rate may be particularly advantageous where fixed window machine learning algorithms are used, e.g., where the feature extraction process above uses a fixed window CNN. Varying the sample rate proportionally with heart rate ensures that the same number of full ECG beats are present in said fixed sample window. In effect, such adaptive filtering normalises the ECG signals, thereby factoring out any variations due to heart rate before the ECG signal is passed to the ML algorithm. This may increase the accuracy of the disclosed identification method. Adaptive filtering may be preferable to other means of reducing undesirable differences resulting from heart rate variation. For example, it would be possible to train the machine learning model using ECG signals at different heart rates, imposing a penalty function to ensure that heart rate variation does not impact the resulting vector representations. But this would significantly increase the training demand.

The sampling of each ECG signal may be configured so that at least 2000 samples are provided per three full ECG cycles.

The aforementioned fixed sample window may be at least 1000 samples, at least 2000 samples or at least 5000 samples, for example. The adaptive sampling rate may be configured so that a fixed sample window contains at least 1, at least 3, at least 5 or least 10 full ECG beats, for example. The adaptive sampling frequency may be configured to ensure that a minimum sampling frequency is at least that of an expected Nyquist frequency for sampling ECG signals. The Nyquist frequency may be 100 Hz, 200 Hz or 300 Hz, for example.

The heart rate may be determined from a measurement of an R-R peak interval in the ECG signal.

The R-R peak internal may be a suitable feature to use for heart rate determination, given the typical prominence of the R peaks in an ECG signal. The R peaks, and the respective R-R interval, may be identified in the ECG signal using the feature extraction algorithm discussed above.

The sampling of the ECG signal may be performed as a post-processing step following the ECG signal being obtained.

The sampling of the ECG signal may be implemented at the acquisition stage using hardware; the physical rate at which ECG sensors measure an ECG signal of a driver and/or the rate at which the ECG signal value is recorded. However, adaptive acquisition may mean that some information in the ECG signal, e.g., the heart rate, is lost. Alternatively, the sampling of the ECG signal may be done following acquisition to ensure that the heart rate can be determined. The ECG signal may be obtained as an analogue signal, for example, with analogue to digital converters being used to sample the ECG signal at a specified sample rate. Acquisition may be performed at a fixed, higher sampling rate (e.g., 2.5 kHz) to ensure that the sufficient information is captured, with the ECG signal then being re-quantised to a lower, adaptive sample rate. Any suitable re-quantization process, such as interpolation, may be used to provide the adaptive sample rate.

The measured heart rate may be provided as an input to the machine learning algorithm when obtaining the corresponding vector representation of the ECG signal.

Although it may be desirable to make the ECG signal heart rate invariant prior to the application of ML algorithms, the heart rate may be an important characteristic for ECG classification. Thus, the heart rate may be provided as an input to the ML algorithm (e.g., alongside the extracted peaks, as discussed above). The ML algorithm may be trained using said heart rate input, thus allowing analysis of heart-rate dependent ECG features that may be useful for identifying drivers. The heart rate input that is provided to the ML algorithm may be an average heart rate of the driver during the acquisition of the ECG signal, for example.

The comparison of the second vector representation to the labelled first vector representation may comprise determining a distance in a vector space between the two vector representations. The similarity metric may be a measure of said distance.

The distance between the first and second vector representation may provide a suitable measure of similarity and may be determined using a suitable function. The distance may be measured as a cosine similarity, a Euclidean distance or a Manhattan distance in the vector space, for example. The predetermined threshold against which the similarity metric is compared may likewise be distance in the vector space. The threshold may be a fixed value (i.e., a predetermined value that is considered to be an upper limit with which vector representations are considered to be a match). Alternatively, the threshold may be a variable function, for example based on the variance of the first vector representation space between the plurality of first vector representations.

According to a second aspect, there is provided a system for determining an identity of a driver. The system comprises: an identity token, the identity token comprising information identifying a driver; at least one electrocardiogram, ECG, electrode configured to be contacted by a body part of a driver and to output an ECG signal; an embedding module comprising a machine learning algorithm, the embedding module being configured to receive at least part of the ECG signal and to use the machine learning algorithm to obtain a vector representation of features of the first ECG signal; and a classifier, the classifier being configured to compare two vector representations to determine a similarity metric.

When used according to the present disclosure, the system is configured to: obtain, using the at least one ECG electrode, a first electrocardiogram, ECG, signal for an authorised driver; provide at least part of the first ECG signal to the machine learning algorithm of the embedding module to obtain a first vector representation of features of the first ECG signal; label the first vector representation as belonging to the authorised driver, the labelling being based upon an identity token provided by the authorised driver prior to or at the time of obtaining the first ECG signal; obtain, using the at least one ECG electrode, a second ECG signal for a candidate driver; provide at least part of the second ECG signal to the machine learning algorithm of the embedding module to obtain a second vector representation of features of the second ECG signal; compare, using the classifier, the second vector representation to the labelled first vector representation to determine a similarity metric; and if the similarity metric is within a predetermined threshold, provide confirmation that the candidate driver is the authorised driver.

The system may be configured to perform the method according to the first aspect.

The at least one ECG electrode may comprise three ECG electrodes disposed on a steering wheel of a vehicle and/or vehicle controls mounted on a steering wheel column of a vehicle.

Using three ECG electrodes to capture multiple ECG leads may enable better quality ECG signals to be obtained, compared to using only two ECG electrodes (e.g., the ECG signal may have lower signal to noise ratio). Disposing the ECG electrodes on the steering wheel of the vehicle and/or on vehicle controls mounted on the steering wheel column of the vehicle can allow for ECG signals to be obtained when the driver is sat in a natural driving position and gripping the steering wheel (e.g., having just entered and sat down in a driver's seat of the vehicle) and/or while the driver is actively driving the vehicle. It may be preferable for all the ECG electrodes to be disposed on the steering wheel, so that ECG signals can be obtained from the driver at all times while they are holding the steering wheel. However, some or additional ECG electrodes may be provided on other control surfaces, e.g., on gear shift control paddles located behind/next to the steering wheel.

A first and second ECG electrode may be disposed on a front surface of the steering wheel so that each of the first and second ECG electrodes is contactable by one of the driver's thumbs when gripping the steering wheel. A third ECG electrode may be disposed on a rear surface of the steering wheel so that the third ECG electrode is contactable by one of the driver's fingers when gripping the steering wheel.

This arrangement may be preferable in allowing the driver to contact all three electrodes at the same time while still gripping the steering wheel with both hands in a natural driving position (e.g., with hands at “10 and 2” or at “9 and 3” on the steering wheel). The first and second ECG electrodes may be disposed on spokes of the steering wheel and/or on the outer circumference of the steering wheel facing the driver, so that the driver can contact the first and second electrodes with their left and right thumb respectively. The third ECG electrode may be disposed on spokes of the steering wheel and/or on the outer circumference of the steering wheel facing away from the driver, so that one of the driver's fingers can curve around the steering wheel and contact the third electrode on the rear side of the steering wheel.

The identity token may be a key for the vehicle or a mobile phone that is configured to communicate with the vehicle.

Many modern vehicle keys are capable of communicating with the vehicle (e.g., to enable keyless entry or starting of the vehicle). Likewise, may modern vehicle manufacturers provide phone applications for engaging with the vehicle (e.g., for providing remote control of the vehicle). The vehicle key and/or the mobile phone may communicate with the vehicle using any wireless protocol, for example Bluetooth of Wi-Fi. A vehicle key or a mobile phone may each constitute a suitable identity token; possession of the vehicle key or the mobile phone may be regarded as an indication that the driver is the owner of said key/mobile phone. In the case of a mobile phone, the driver may be prompted to provide additional identification. For example, the driver may provide a passcode or fingerprint to the mobile phone after being prompted to do so, thereby confirming that they are the authorised driver associated with this identity token. A key may be more likely to be shared between different users than a mobile phone: for example in the case of husband and wife sharing a family car and a family vehicle.

According to a third aspect, there is provided a vehicle comprising the system of the second aspect. The vehicle may be any vehicle, including but not limited to a car, a van, a lorry or an agricultural vehicle.

According to a fourth aspect, there is provided a non-transitory computer-readable medium comprising instructions which, when executed by a computer, cause the computer to: obtain a first electrocardiogram, ECG, signal for an authorised driver; provide at least part of the first ECG signal to a machine learning algorithm to obtain a first vector representation of features of the first ECG signal; label the first vector representation as belonging to the authorised driver, the labelling being based upon an identity token provided by the authorised driver prior to or at the time of obtaining the first ECG signal; obtain a second ECG signal for a candidate driver; provide at least part of the second ECG signal to the machine learning algorithm to obtain a second vector representation of features of the second ECG signal; compare the second vector representation to the labelled first vector representation to determine a similarity metric; and if the similarity metric is within a predetermined threshold, provide confirmation that the candidate driver is the authorised driver.

The non-transitory computer-readable medium may comprise instructions which, when executed by a computer, cause the computer to carry out a method according to the first aspect.

The first, second, etc. features may be the same features for each of the authorised/candidate drivers. Each vector representation obtained from the machine learning algorithm may represent the same one or more features for each respective ECG signal.

These and other aspects of the disclosure will be apparent from, and explained with reference to, the embodiments described hereinafter.

### BRIEF DESCRIPTION OF THE DRAWINGS

Embodiments of the invention will be described, purely by way of example, with reference to the accompanying drawings, in which:

FIG. 1 shows an example of a system for determining an identity of a driver;

FIG. 2 shows further details of a system for determining an identity of a driver;

FIG. 3 shows an example of an ECG signal from which features have been extracted in accordance with the present disclosure; and

FIG. 4 shows an example of a method for comparing ECG vector representations in accordance with the present disclosure.

It should be noted that the Figures are diagrammatic and not necessarily drawn to scale. Relative dimensions and proportions of parts of these Figures may have been shown exaggerated or reduced in size, for the sake of clarity and convenience in the drawings. The same reference signs are generally used to refer to corresponding or similar features in modified and different embodiments.

### DETAILED DESCRIPTION

Referring to FIG. 1, a schematic diagram of an example system 100 for determining an identity of a driver is shown. The system 100 may be incorporated into a vehicle, for example a car (not shown).

The system comprises a plurality of ECG electrodes 110a-c arranged on a steering wheel 101 of the vehicle. In the example system 100, there are three ECG electrodes. A first ECG electrode 110a is disposed on the left-hand side of a front surface of the steering wheel 101 (i.e., the side of the steering wheel 101 facing the driver) across part of the outer circumference of the steering wheel and a spoke 102 of the steering wheel. A second ECG electrode 110b is similarly disposed on the right-hand side of the front surface of the steering wheel 101. A third ECG electrode is provided on a rear surface of the steering wheel 101 (i.e., the side of the steering wheel 101 facing away the driver), for example across part of the outer circumference of the steering wheel and the steering wheel spoke 102.

When the driver sits in a driver's seat of the vehicle, they may grip the steering wheel 101 with both hands in an ordinary driving position. In doing so, the driver's left thumb may contact the first ECG electrode 110a, their right thumb may contact the second ECG electrode 110b, and one of their right-hand fingers may contact the third ECG electrode 110c. Thus, three lead ECG measurements can be taken of the driver while they are holding the steering wheel (discussed further below). The ECG signals obtained by the ECG electrodes are provided to an ECG processor 200, for example using wires running within/along the surfaces of the steering wheel 101. The ECG signals of the driver may then be analysed at the ECG processor 200 according to the methods of the present disclosure.

The ECG electrode 110a-c may be any commercially available ECG electrode. Each ECG electrode 110 may comprise an electrically conductive surface (e.g., a steel plate) against which the driver places their skin. Each ECG electrode 110 may be a ‘wet’ electrode—i.e., comprising an electrically conductive liquid or gel so as to increase the electrical contact between the driver's skin and the ECG electrode. Each ECG electrode 110 may be of a modular component; there may be an electrode base which is attached to the steering wheel and connected to wires/leads which couple the ECG electrode to the ECG processor 200, and their may be an electrode surface which is electrically coupled to and detachable from the electrode base. The electrode surface may thus be replaced if it becomes damaged or worn.

The system further comprises an identity token 120. The identity token may be configured to communicate with the vehicle and the ECG processor 200 wirelessly. In this example, the identity token 120 is shown as a mobile phone. However, the identity token 120 may be another physical device, such as a key for the vehicle. The identity token 120 may be presented to the vehicle and ECG processor 200 when the driver interacts with the vehicle. For example, a mobile phone or vehicle key may be used to unlock and/or start the vehicle. Because the identity token 120 communicates with the vehicle/ECG processor, the ECG processor 200 is aware that a driver associated with the identity token 120 is using the vehicle.

The identity token 120 provides identity information regarding the custodian of the identity token 120 (which may be assumed to be the driver) to the ECG processor 200. This identity information may be used to label or infer the ECG signals obtained from the driver, as discussed further below. Where the identity token 120 is a mobile phone, this identity information may be specific to a particular person. For example, the mobile phone may comprise a mobile phone application associated with the vehicle, which the driver has logged into and provided personal information (e.g., a name and/or contact details). This information may be made available to the ECG processor 200—e.g., when the mobile phone of person X is presented to the ECG processor 120, the ECG processor is aware that the current driver is person X. Where the identity token is a vehicle key, the identity information may only be an identity of said vehicle key—i.e., the ECG processor may only be aware that the driver is using vehicle key A.

The use of mobile phones as identity keys (or other similar electronic devices which contain personal information) may be advantageous for the methods of the present disclosure. Where each driver has their own mobile phone, the ECG processor 200 may be provided with individual identity information for each driver, which may assist in generating individualised ECG profiles for each driver (discussed further below). By contrast, where the identity token 120 is a vehicle key, this key may be shared between drivers. Nonetheless, individualised ECG profiles may still be provided on a key-by-key basis—e.g., a first vehicle key A may be regarded as belonging to a first person X, while a second vehicle key B may be regarded as belonging to a second person Y.

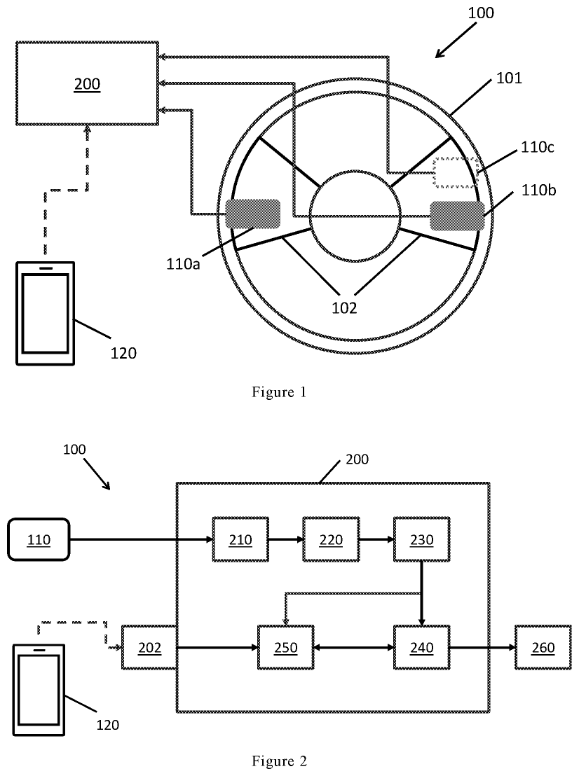

Referring to FIG. 2, a schematic diagram of an example system 100 for determining an identity of a driver is shown. The system 100 may be implemented as shown in FIG. 1 (i.e., within a vehicle steering wheel). As in FIG. 1, the system 100 comprises one or more ECG electrodes 110, an identity token 120 and an ECG processor 200.

The ECG processor 200 receives an ECG signal from the one or more ECG electrodes 110. The ECG processor 200 may comprise a pre-processing unit 210 that receives a raw ECG signal from the ECG electrode 110.

The pre-processing unit 210 may comprise an analogue front end (AFE) that is configured to receive, amplify and filter the raw ECG signal. Any suitable AFE may be used. In one example, the AFE is an AD8232 from Analogue Devices which supports three electrode configurations like those discussed above. The AFE may include an amplifier, adjustable low-pass and high-pass filters and DC common mode rejection. An ECG signal may typically be expected to comprise signals in the range of 0.5 Hz to 150 Hz, and so low and high-pass filters may be applied accordingly. Additionally, filtering may also be applied to remove any expected noise frequencies. For example, a 50 Hz notch filter may be applied to remove electrical line noise. Any suitable filtering may be applied by the AFE; filters may include smoothing via first order, infinite impulse response (IIR) filters, sliding window median filters, Wiener filters, notch filters, and/or high pass and band pass Butterworth filters. The filtered signal may be clamped; i.e., extremal signal values discarded. Applying filters to the raw ECG signal can lead to numerical instabilities. For example, if there are extremal samples (e.g., caused by noise) in the signal, applying Butterworth filters causes signal artefacts. Additionally, or alternatively, a median filter may be applied to the raw ECG signal first to alleviate transient extremal samples.

The pre-processing unit 210 may also be configured to sample the raw ECG signal. The filtered or raw ECG signal may be passed to an analogue to digital converter (ADC). The ADC may perform an initial sampling at a high frequency rate so as to provide a more useable digital signal. In one example, the ADC samples at a rate of 500,000 samples per second with a bit depth of 12 bits per sample.

The pre-processing unit 210 may further be configured to provide heart-rate adaptive sampling, wherein the sampling rate is adapted based upon a heart rate of the driver as determined from the ECG signal. Adaptive sampling may remove heart rate dependence from subsequent feature extraction and may enable ECG samples of the signal to be provided to a fixed sample window convolutional neural network (CNN) architecture model, as discussed further below.

For example, a fixed-window CNN model may require inputs with 2000 samples, placing a constraint on the pre-processing. An additional constraint is imposed by the Nyquist sampling frequency as determined by the greatest diagnostically relevant frequency component of the ECG signal. For typical ECG signal, the sampling frequency may need to be at least 200 Hz (i.e., an inter-sample interval of 5 ms).

For example, consider an ECG signal of a subject with a heart rate of 80 BPM sampled at 200 Hz. Using a 2000 sample window, approximately 13 full ECG cycles/beats would be obtained during a 10 second acquisition time. However, where the subject's heart rate increases to 130 BPM, the same 10 second/2000 sample acquisition window will contain almost 22 full ECG cycles. In this scenario the number of cycles is nearly doubled and thus the samples per cycle (the signal resolution) is nearly halved.

The number of ECG cycles Nₙ in a window of n samples is generally given by

$${N}_{n} = \frac{n}{{f}_{s}} \frac{{F}_{HR}}{60} \qquad ( 1 )$$

where fₛ is the sampling frequency in Hertz (samples per second) and F_{HR} is heart rate in BPM. Heart rate is a known natural, variation in the ECG signal that can be expected. The feature extraction/classification model should be heart rate invariant, so that changes in driver heart rate do not result in different identification results.

The sample frequency fₛ may be varied proportionally to the driver's heart rate so that variations due to heart rate are removed from the ECG signal windows. This will let the system preserve a consistent, heart-rate-agnostic ECG signal resolution. The expected 200 Hz Nyquist frequency may be imposed as a lower bound to the sampling frequency. Assuming that a lowest possible heart rate is 20 BPM, and setting the base sampling frequency to be 200 Hz, a 2000 sample window will contain 3 full ECG cycles. Rearranging equation 1 to find the sampling frequency fₛ where the number of cycles Nₙ is 3 and the number of samples nis 2000, the adaptive sampling frequency is

$${f}_{s} = \frac{n}{{N}_{n}} \frac{{F}_{HR}}{60} = \frac{2000}{3} \frac{{F}_{HR}}{60} \approx 11 \cdot {F}_{HR} \qquad ( 2 )$$

Using this adaptive sampling result and the examples above, the 80 BPM signal will be sampled at 880 Hz while the 130 BPM signal is sampled at 1430 Hz. In both cases the 2000 sample window will contain 3 full ECG cycles. Assuming a maximum heart rate of 220 BPM, the maximum sampling frequency required from the system will be 2.42 kHz.

The adaptive sampling could be performed at the acquisition stage (in hardware) or as a data-processing step at the pre-processing unit 210 (in software). In order to retain information regarding the heart rate, adaptive sampling at the pre-processing unit 210 may be preferable. For simplicity and downstream compatibility, a fixed acquisition frequency may be used (e.g., at 2.5 kHz) with the adaptive sampling being applied to the acquired ECG signal for re-quantisation.

The heart rate used in equation 2 to determine the sampling frequency may be determined based on the ECG signal. The R-R interval of the ECG signal may be identified, with the heart rate given by 60 divided by the R-R interval in seconds. The R-R interval may be found using any suitable means. A feature extraction model, as discussed further below, may be applied to the ECG signal to identify the R peaks. Alternatively, given that the R peaks are usually most prominent in the ECG signal, a simple highest peak finding algorithm may be used. The average of multiple R-R intervals (e.g., for the three full cycles in the fixed window discussed above) may be used to determine heart rate.

The pre-processing unit 210 thus outputs a pre-processed ECG signal. The pre-processed ECG signal may be provided to a feature extractor 220. The feature extractor 220 is configured to extract features from the ECG signal and output one or more features that may be useful for ECG-based driver identification.

The feature extractor 220 may comprise a machine learning model that has been trained to identify the locations of the PQRST wave peaks in an ECG signal segment. From this, intervals between the peaks may be determined also. The peak locations and inter-peak intervals may comprise extracted features that are provided to the embedding module. The feature extractor machine learning model may have a CNN architecture and configured to receive signal segments with a fixed number of samples. The feature extractor machine learning model may be as defined in WO 2019/234458 A1, the contents of which is incorporated herein by reference.

The extracted ECG output features are provided to an embedding module 230. The embedding module 230 comprises a machine learning algorithm that is configured to produce an embedding vector (also referred to as a vector representation) that represents the features of the ECG signal in question. A single vector representation can be understood to be a single point in a vector space—i.e., the vector representation has a magnitude value in each dimension of the multidimensional vector space. However, where multiple ECG signals or multiple ECG signal segments are provided to the ECG processor, discussed further below, a vector representation may be determined for each of the ECG signals/segments. The plurality of vector representations may be combined (e.g., an average taken) and the variance between the vector representations calculated. Thus, the plurality of vector representations may be combined into a single vector representation space; a volume within the vector space centred around an average vector representation and extending in each dimension by some distance as given by the determined variance.

In addition to the features extracted from the ECG signal provided to the embedding module 230, the heart rate (as determined above, e.g., using the R-R peak interval) may be provided as an additional input to the embedding module 230. Therefore, although the ECG signal has been made heart-rate invariant through the use of adaptive filtering, heart rate dependent ECG features may still be accounted for in the embedding.

The vector representation (i.e., the embedding vector of the ECG signal/extracted ECG features) is provided to a classifier 240. The classifier 240 is configured to compare the vector representation as determined for the present ECG signal against previous vector representations that have been obtained from prior ECG signals, in order to determine a similarity metric between the two. The previous vector representations may be stored in a memory 250 and retrieved by the classifier 240.

When the prior ECG signals were obtained, an identity token 120 for the driver in question may have been present. The ECG processor 200 may comprise a wireless communications module 202 for communicating with the identity token 120, such that identity information regarding the driver can be provided to the ECG processor 200 alongside the ECG signals. Where the identity token 120 is a mobile phone, this identity information may include the name of the driver, said name having been provided to/stored in a mobile application by the driver previously. The identity information obtained from the identity token 120 may be used to label the representations vectors within the memory 250—i.e., the vector representation may be saved in the memory 250 as belonging to the driver who has presented the identity token 120.

The comparison of the current vector representation to previous vector representations may comprise calculating a vector distance between the two said vectors. For example, a vector representation space may have been obtained (using multiple vector representations as above) for a known, authorised driver and stored in the memory 250. A new vector representation as determined for a present ECG signal is compared against the vector representation space and a distance between the two computed. The computed distance is compared to a threshold distance. If the computed distance between the two vectors is less than the threshold distance, then the current vector representation is considered to ‘match’ the previous vector representations/vector space and the driver providing the present ECG signal (a candidate driver) is determined to be the authorised driver. If the computed distance between the two vectors is greater than the threshold distance, the vectors are not considered to match. The threshold distance may be a fixed value (i.e., some predetermined similarity threshold). Alternatively, the threshold distance may be based upon the variance of the vector representation space (e.g., does the current vector representation fall within the previous vector representation pace).

Based upon the outcome of the comparison, the classifier 240 may output a similarity indication 260, e.g., an indication of whether the present ECG signal matches previous ECG signals (and thus whether the candidate driver is considered to be the authorised driver). The vehicle within which the system 100 is implemented may be configured to perform further operations based on the similarity indication 260. For example, where the ECG signals do match, the driver may be granted additional functionality over the vehicle. Where the ECG signals do not match, the system 100 may instead rely upon the identity tokens 120 alone—e.g., does the identity token 120 presented by the candidate driver match an identity token used previously.

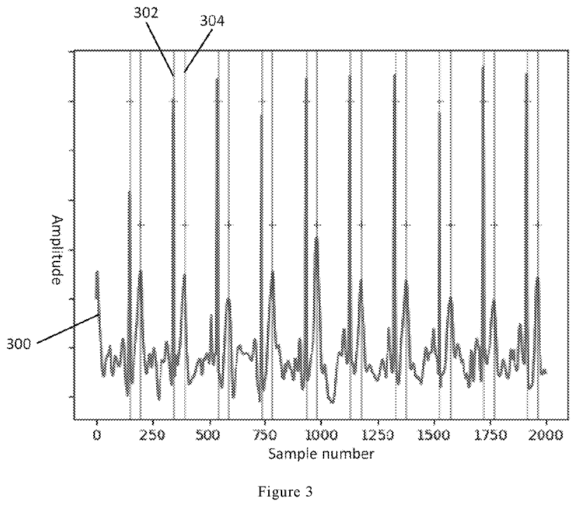

Referring to FIG. 3, an example of an ECG signal 300 is shown that has been subject to some of the pre-processing and feature extraction techniques discussed above. In the example of FIG. 3, the ECG signal 300 has been subjected to a Butterworth bandpass signal with a window of 0.5 to 150 Hz window and a notch filter centred around 50 Hz. A 2000 sample window segment of the filtered ECG signal, in this case comprising 10 full cycles, was the passed to a fixed-window CNN model for feature extraction, as discussed above. In FIG. 3, the identified R peaks 302 and the T peaks 304 are shown with vertical lines for each of the beats.

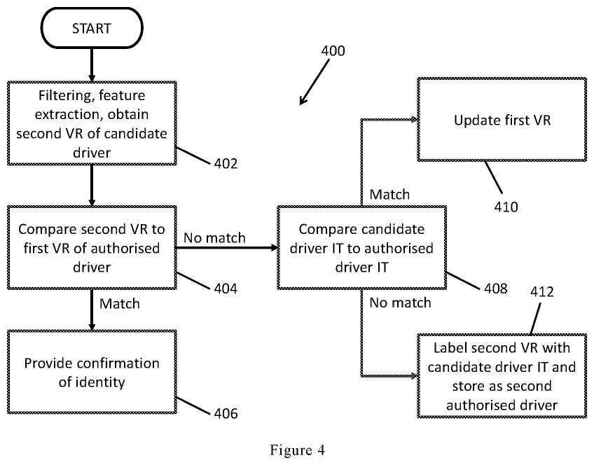

Referring to FIG. 4, a schematic flow diagram is shown illustrating an example method for determining driver identity based upon ECG signals. The flow diagram of FIG. 4 shows a process whereby a newly obtained ECG signal for a candidate driver is compared to ECG signals previous obtained from an authorised driver. Thus, prior to the method shown in FIG. 4, a first use of the system (otherwise referred to as an initial onboarding) is performed.

When a driver uses the system for the first time, there is no information about the driver's biometrics. At this stage, the system will primarily rely upon existing authentication methods (e.g., the identity token mobile phone or vehicle key) to identify and authorise the driver to use the vehicle. However, this also means that there is no expectation from the driver that the ECG-based identification process will work without an initial onboarding process. During the first use/initial onboarding, the driver may be instructed to place their thumbs/fingers on the ECG electrodes in order to obtain a first ECG signal. This initial acquisition may occur over an extended period once the driver has entered the vehicle—e.g., the driver may be instructed to place their thumbs/fingers on the ECG electrodes for 10 to 15 minutes. This may be done prior to and while the driver is operating the vehicle. Additionally, the driver may be instructed to reposition their thumbs/fingers on the electrode, but this may also occur naturally during driving.

Thus, during this initial onboarding, the driver provides an extended ECG signal to the ECG processor that preferably includes some variance/noise. As discussed above, a single ECG signal segment of fixed sample window may contain three full ECG cycles/beats and thus be a few seconds in length. Therefore, the 10-15 minute initial onboarding may contain many ECG signal segments. Each of these may be processed independently using the aforementioned methods, therefore providing many vector representations. The vector representations may be combined using the aforementioned methods, thereby producing a vector representation space for the authorised driver. The vector representation space may be labelled as belonging to the authorised driver based upon the identity token presented by the authorised driver and stored in a memory for future comparison. The labelled, stored vector representation space can be regarded as an ‘ECG profile’ that identifies the authorised driver.

Following this initial onboarding, the comparison process 400 as illustrated in FIG. 4 can be commenced with. A candidate driver provides a new (e.g., a second) ECG signal. This new ECG signal may undergo the same pre-processing, feature extraction and embedding process as discussed above in order to obtain 402 a second vector representation (VR) for the candidate driver's ECG signal. The second vector representation is then compared 404 to the previously obtained and stored vector representation of the authorised driver (or vector representation space, assuming that the authorised driver provided multiple ECG signal samples during the aforementioned initial onboarding). As above, this this comparison may comprise computing a distance between the vectors in order to obtain a similarity metric (e.g., a distance) that is compared to a threshold.

If the similarity metric is considered to indicate a match between the second vector representation of the candidate driver and the vector representation space of the authorised driver (e.g., the computed vector distance is less than a threshold), the system may provide 406 confirmation that the candidate driver is the authorised driver. Even where a match is confirmed, this new ECG signal/vector representation may be used to update the driver's ECG profile as described below in step 410 (not shown in FIG. 4).

If the similarity metric is considered to indicate that there is no match between the second vector representation of the candidate driver and the vector representation space of the authorised driver (e.g., the computed vector distance is greater than a threshold), the system may instead rely upon separate identity tokens, such as a mobile phone for a vehicle. Assuming that the candidate driver has provided a suitable identity token, this may be used to operate the vehicle, for example. Additionally, the identity token (IT) of the candidate driver (the second identity token) is compared 408 to the identity token of the authorised driver (the first identity token) that was previously presented by the authorised driver and used to label the stored/first vector representation space. The identity token, or identity information provided therefrom, may be stored alongside the vector representation space. For example, where the identity tokens are mobile phones comprising applications related to the vehicle, a driver name provided by the candidate driver in said mobile phone application may be compared to a driver name provided by the authorised driver in their mobile phone application.

If the identity token of the candidate driver matches the identity token of the authorised driver, this may be taken as meaning that the candidate driver and the authorised driver are in fact the same person, but that the ECG-based comparison 404 failed to match the candidate driver to the authorised driver. In such an instance, the first vector representation/vector representation space of the authorised driver, previously obtained and stored in the initial onboarding, may be updated 410 to include the new, second vector presentation. The updating 410 of the vector representation space may comprise combining the first and second vector representations, as discussed above.

If the identity token of the candidate driver does not match the identity token of the authorised driver, this may be taken as meaning that the candidate driver is a new driver who has not previously been saved to the system as an authorised driver. However, by virtue of having a suitable identity token, the candidate driver may be regarded as being authorised to operate the vehicle. Therefore, the second vector representation of the candidate driver may be labelled using the second identity token and stored 412 in memory as a new, second authorised driver. The handling, labelling and storing of the candidate's second vector representation may be done similarly to the first vector representation as discussed above—e.g., ECG signals may be obtained over an extended duration so as to provide many second vector representations, which may be combined into a second vector representation. Thus, step 412 can be regarded as an initial onboarding of the candidate driver (i.e., establishing a new ECG profile for the candidate as a new, second authorised driver).

The process shown in FIG. 4 may be utilised repeatedly. E.g., the comparison process 400 may be invoked each time a driver enters the vehicle and provides further ECG signals. Thus, the driver's identity can be confirmed each time; the authorised driver's ECG profile/vector representation space can be updated with each new ECG signal provided; and/or additional authorised drivers can be added to the system each time a new candidate driver presents themselves.

An ideal ECG-based authentication system should be able to carry out one-shot learning with a single, known sample segment of a given driver's ECG signal and subsequently be able to identify that driver given a new ECG signal. However, compared to clinical ECG (e.g., acquired 444 through electrodes on the chest), the in-vehicle ‘real world’ ECG signals of the present system, acquired through the thumbs/fingers may be noisier and more unstable. Signal deviation from the true driver ECG signal can obscure key characteristics of the ECG signal that are unique to each driver, which can make the process of learning to discern a driver's identity through ECG challenging.

However, the method of the present disclosure can provide a staggered, self-learning onboarding process for ECG-based driver identification. As described above, where it is found that the comparison of the candidate driver and authorised driver ECG has failed to identify a match, the system automatically updates the authorised driver's ECG profile to include the new vector representations. The process shown in FIG. 4 may be repeated until the variance in the authorised driver's ECG is captured in the driver's vector representation space (i.e., a domain in an embedding space). At this point, any subsequent invocations of the process with a new ECG signal from the same driver should correctly be able to identify the driver, as per the confirmation provided at step 406. Thus, the system may grant the driver access to the vehicle/vehicle operations without any secondary authentication methods—e.g., the driver may be able to use the vehicle even without presenting their identity token (such as when they have forgotten their mobile phone.

Likewise, the method of the present disclosure is able to provide automatic enrolment of new, further authorised drivers. Each time a new driver interacts with the system, they may undergo the initial enrolment process discussed above in order to establish an ECG profile/vector representation space. Thus, overtime, the system can learn to identify multiple drivers.

It will be appreciated that any of the methods described herein, and any step of the methods, can be implemented by a computer. Such implementation may take the form of a processor executing instructions stored on a non-transitory computer-readable medium or media, wherein when executed the instructions cause the processor to perform any one or more steps of any of the methods described herein. Individual steps of any method may be implemented by different processors that are all collectively acting in accordance with computer-readable instructions stored on one or more storage media. The processor(s) may be component(s) of system, for example a processor of a device.

As used herein, the term “non-transitory computer-readable media/medium” is intended to be representative of any tangible computer-based device implemented in any method or technology for short-term and long-term storage of information, such as, computer-readable instructions, data structures, program modules and sub-modules, or other data in any device. The methods described herein may be encoded as executable instructions embodied in a tangible, non-transitory, computer readable medium, including, without limitation, a storage device, and/or a memory device. Such instructions, when executed by a processor, cause the processor to perform at least a portion of the methods described herein. Furthermore, as used herein, the term “non-transitory computer-readable media/medium” includes all tangible, computer-readable media, including, without limitation, non-transitory computer storage devices, including, without limitation, volatile and non-volatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

As will be appreciated based on the specification herein, the above-described embodiments of the disclosure may be implemented using computer programming or engineering techniques including computer software, firmware, hardware or any combination or subset thereof. Any such resulting program, having computer-readable code means, may be embodied, or provided within one or more computer-readable media, thereby making a computer program product, i.e., an article of manufacture, according to the discussed embodiments of the disclosure. The article of manufacture containing the computer code may be made and/or used by executing the code directly from one medium, by copying the code from one medium to another medium, or by transmitting the code over a network.

From reading the present disclosure, other variations and modifications will be apparent to the skilled person. Such variations and modifications may involve equivalent and other features which are already known in the art of coupled inductors, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub combination. The applicant hereby gives notice that new claims may be formulated to such features and/or combinations of such features during the prosecution of the present application or of any further application derived therefrom.

For the sake of completeness, it is also stated that the term “comprising” does not exclude other elements or steps, the term “a” or “an” does not exclude a plurality. Reference signs in the claims shall not be construed as limiting the scope of the claims.

What is claimed is:

1. A system for determining an identity of a driver, the system comprising:
an identity token comprising information identifying the driver;
at least one electrocardiogram (ECG) electrode configured to be contacted by a body part of the driver and to output an ECG signal;
an ECG processor comprising an embedding module, the embedding module comprising a machine learning algorithm, the embedding module being configured to receive at least part of the ECG signal and to use the machine learning algorithm to obtain a vector representation of features of the ECG signal; and
a classifier configured to compare two vector representations to determine a similarity metric;
wherein the system is configured to:
receive a first identity token from an authorised driver;
obtain, at the time of or after receiving the first identity token, a first ECG signal for the authorised driver;
provide at least part of the first ECG signal to the machine learning algorithm to obtain a first vector representation of first features of the first ECG signal;
label the first vector representation as belonging to the authorised driver, the labelling being based upon the first identity token;
obtain a second ECG signal for a first candidate driver;
provide at least part of the second ECG signal to the machine learning algorithm to obtain a second vector representation of second features of the second ECG signal;
compare the second vector representation to the labelled first vector representation to determine the similarity metric; and
if the similarity metric is within a predetermined threshold, provide confirmation that the first candidate driver is the authorised driver.

2. The system of claim 1, wherein the at least one ECG electrode comprises three ECG electrodes configured to be disposed on a steering wheel of a vehicle and/or vehicle controls mounted on a steering wheel column of the vehicle.

3. The system of claim 2, wherein a first ECG electrode and a second ECG electrode of the three ECG electrodes are configured to be disposed on a front surface of the steering wheel so that the first ECG electrode and the second ECG electrode are contactable by thumbs gripping the steering wheel, and wherein a third ECG electrode of the three ECG electrodes is configured to be disposed on a rear surface of the steering wheel so that the third ECG electrode is contactable by a finger when gripping the steering wheel.

4. The system of claim 3, wherein the first ECG electrode, the second ECG electrode, and the third ECG electrode are disposed on spokes of the steering wheel and/or on an outer circumference of the steering wheel.

5. The system of claim 2, wherein the first identity token is a key for the vehicle that is configured to communicate with the vehicle.

6. The system of claim 2, wherein the first identity token is a mobile phone that is configured to communicate with the vehicle.

7. The system of claim 2, wherein the first identity token is configured to communicate with the vehicle using a wireless protocol.

8. The system of claim 2, further comprising the vehicle.

9. A computer implemented method of determining an identity of a driver, the computer implemented method comprising:
receiving a first identity token from an authorised driver;
obtaining, at the time of or after receiving the first identity token, a first electrocardiogram (ECG) signal for the authorised driver;
providing at least part of the first ECG signal to a machine learning algorithm to obtain a first vector representation of first features of the first ECG signal;
labelling the first vector representation as belonging to the authorised driver, the labelling being based upon the first identity token;
obtaining a second ECG signal for a first candidate driver;
providing at least part of the second ECG signal to the machine learning algorithm to obtain a second vector representation of second features of the second ECG signal;
comparing the second vector representation to the labelled first vector representation to determine a similarity metric; and
if the similarity metric is within a predetermined threshold, providing confirmation that the first candidate driver is the authorised driver.

10. The computer implemented method of claim 9, wherein the first ECG signal and the second ECG signal are obtained via at least one ECG electrode, and wherein the at least one ECG electrode comprises three ECG electrodes disposed on a steering wheel of a vehicle and/or vehicle controls mounted on a steering wheel column of the vehicle.

11. The computer implemented method of claim 10, wherein a first ECG electrode and a second ECG electrode of the three ECG electrodes are disposed on a front surface of the steering wheel so that the first ECG electrode and the second ECG electrode are contactable by thumbs gripping the steering wheel, and wherein a third ECG electrode of the three ECG electrodes is disposed on a rear surface of the steering wheel so that the third ECG electrode is contactable by a finger when gripping the steering wheel.

12. The computer implemented method of claim 10, wherein the first identity token is a key for the vehicle that is configured to communicate with the vehicle.

13. The computer implemented method of claim 10, wherein the first identity token is a mobile phone that is configured to communicate with the vehicle.

14. The computer implemented method of claim 10, wherein the computer implemented method is implemented by the vehicle.

15. A non-transitory computer-readable medium comprising instructions which, when executed by a computer, cause the computer to:
receive a first identity token from an authorised driver;
obtain, at the time of or after receiving the first identity token, a first electrocardiogram (ECG) signal for the authorised driver;
provide at least part of the first ECG signal to a machine learning algorithm to obtain a first vector representation of first features of the first ECG signal;
label the first vector representation as belonging to the authorised driver, the labelling being based upon the first identity token;
obtain a second ECG signal for a first candidate driver;
provide at least part of the second ECG signal to the machine learning algorithm to obtain a second vector representation of second features of the second ECG signal;
compare the second vector representation to the labelled first vector representation to determine a similarity metric; and
if the similarity metric is within a predetermined threshold, provide confirmation that the first candidate driver is the authorised driver.

16. The non-transitory computer-readable medium of claim 15, wherein the first ECG signal and the second ECG signal are obtained via at least one ECG electrode, and wherein the at least one ECG electrode comprises three ECG electrodes configured to be disposed on a steering wheel of a vehicle and/or vehicle controls mounted on a steering wheel column of the vehicle.

17. The non-transitory computer-readable medium of claim 16, wherein a first ECG electrode and a second ECG electrode of the three ECG electrodes are disposed on a front surface of the steering wheel so that the first ECG electrode and the second ECG electrode are contactable by thumbs gripping the steering wheel, and wherein a third ECG electrode of the three ECG electrodes is disposed on a rear surface of the steering wheel so that the third ECG electrode is contactable by a finger when gripping the steering wheel.

18. The non-transitory computer-readable medium of claim 16, wherein the first identity token is a key for the vehicle that is configured to communicate with the vehicle.

19. The non-transitory computer-readable medium of claim 16, wherein the first identity token is a mobile phone that is configured to communicate with the vehicle.

20. The non-transitory computer-readable medium of claim 16, wherein the instructions are executed by a computer of the vehicle.
